(19) United States
(12) Patent Application Publication
(10) Pub. No.: US 2010/0106225 A1
(43) Pub. Date: Apr. 29, 2010

(54) Transcutaneous Power And/Or Data Transceiver

(75) Inventors: Peter Joseph Ayre, Crows Nest (AU); Martin Christopher Cook, Coogee (AU); John Campbell Woodward, Turramurra (AU); Gregory Peter Hunter, Taren Point (AU); Peter Andrew Watterson, West Ryde (AU)

Correspondence Address: MCCARTER & ENGLISH, LLP STAMFORD, CANTERBURY GREEN, 201 BROAD STREET, 9TH FLOOR, STAMFORD, CT, 06901, US

(73) Assignees: VENTRACOR LIMITED, New South Wales (AU); UNIVERSITY OF TECHNOLOGY SYDNEY, New South Wales (AU)

(21) Appl. No.: 12/650,251

(22) Filed: Dec. 30, 2009

Related U.S. Application Data
(63) Continuation of application No. 10/566,777, filed on Jul. 10, 2006, now abandoned.

(30) Foreign Application Priority Data
Aug. 1, 2003 (AU) 2003904032
Jul. 30, 2004 (AU) PCT/AU2004/001019

(51) Int. Cl. A61F 2/48; A61N 1/08

(52) U.S. Cl. 607/61

(57) ABSTRACT

A device for communicating electric signals across the skin layer (1) of a patient, wherein the device includes: an electrically conductive core (2) capable of forming an EMF flux loop; first (3) and second (4) coils, which are in EMF communication with said electrical conductive core (2) and wherein the first coil (3) is positioned externally to the patient and surrounds at least a first portion of the electrically conductive core (2); and the second coil (4) is implanted beneath or in the skin layer and surrounds at least a second portion of the electrically conductive core.

### FIELD OF INVENTION

The present invention relates to a device and/or a system which is capable of transmitting and/or receiving power and data through the skin or epidermal layer of a patient to an implanted medical device within the body of the patient.

### BACKGROUND

Many advances in medical technology require the use of implantable medical devices to aid, assist or supplement the normal bodily functions of a patient.

Implantable medical devices can be distinguished into two major categories. These categories are: active devices which are devices that actively assist the patient's body and passive devices which passively assist the patient. An active device typically requires a power source, whilst passive devices typically do not require such a source.

An example of an active medical device is an implantable blood pump which actively pumps blood throughout a patient's circulatory system. To accomplish this, the blood pump needs a suitable power supply to function. It also generally requires data control and often returns data to an external controller.

In the past, a percutaneous lead has been used to convey data and power to and from the implantable device. Typically, such a lead would require perforation of the patient's skin layer so as to allow the lead to pass through the skin. These leads usually incorporate features in which the lead may to some extent bond or integrate internally with the patient's body. There are many disadvantages with this type of system. One of these disadvantages is that it creates a site on the patient's skin which is open to infection for long periods of time.

As a result, there has been a long felt need for an improved device that conveys power and/or data to an implantable medical device. It is an object of the present invention to address or ameliorate at least one of the above disadvantages.

### BRIEF DESCRIPTION OF THE INVENTION

The present invention provides for a device for communicating electric signals across the skin layer of a patient, wherein said device includes: an electrically conductive core capable of forming an EMF flux loop; first and second coils, which are in EMF communication with said electrical conductive core and wherein said first coil is positioned externally to said patient and surrounds at least a first portion of said electrically conductive core; and said second coil is implanted beneath or in said skin layer and surrounds at least a second portion of said electrically conductive core.

Preferably, said electrically conductively core is implanted at least partially within said skin layer and said electrically conductively core may be formed in a loop or ring-like configuration. The electrically conductively core may not breach an outer surface of said skin layer.

The electrically conductive core may also be encapsulated within said skin layer.

Additionally, the device may include: a sleeper ring to interact with said first coil; a textured surface on at least a portion of said electrically conductively core; and/or a layer of protective material surrounding at least a portion of the electrically conductive core.

### BRIEF DESCRIPTION OF THE DRAWINGS

Embodiments of the present invention will now be described with reference to the accompanying drawings wherein:

FIG. 1 is a cross sectional view of a first embodiment of the present invention;

FIG. 2 is a cross sectional view of a second embodiment of the present invention;

FIG. 3 is a cross sectional view of a third embodiment of the present invention;

FIG. 4 is a front view of a fourth embodiment of the present invention;

FIG. 5 is a cross sectional side view of the embodiment shown in FIG. 4;

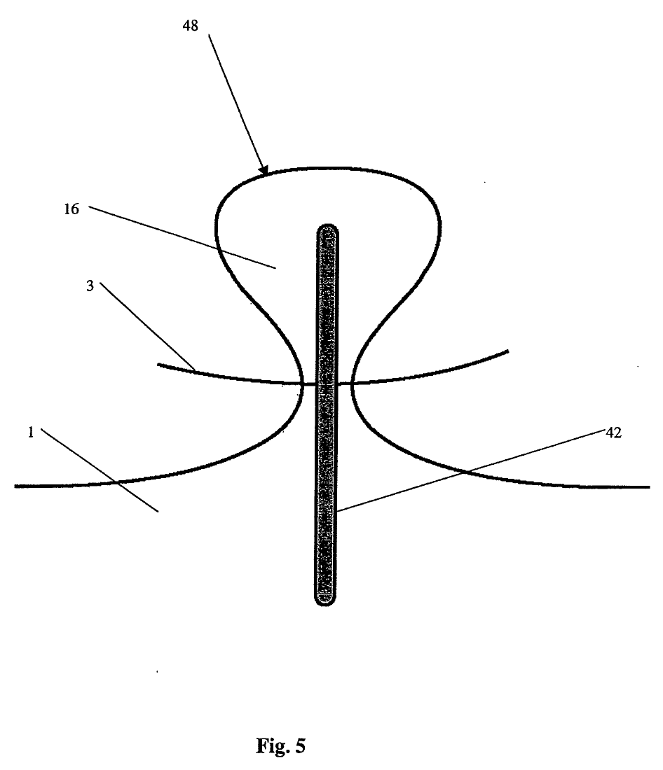

FIG. 6 is a cross sectional view of a fifth embodiment of the present invention;

FIG. 7 is a cross sectional view of a sixth embodiment of the present invention;

FIG. 8 is a cross sectional view of a seventh embodiment of the present invention;

FIG. 9 is a cross sectional view of an eighth embodiment of the present invention; and

FIG. 10 is a cross sectional view of a ninth embodiment of the present invention.

### BRIEF DESCRIPTION OF THE PREFERRED EMBODIMENTS

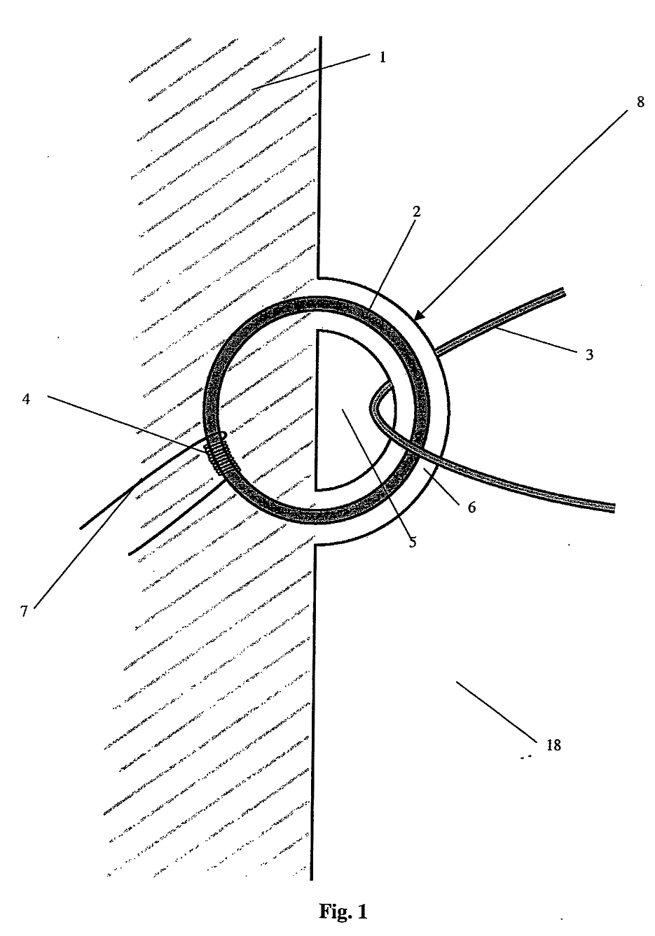

FIG. 1 shows a first embodiment of the present invention. A Transcutaneous Power and/or Data Transceiver System (TPDTS) 8 is shown and comprises an electrically conductive core 2, which in this embodiment is in an annular (or ring-like) configuration.

The electrically conductive core 2 may be partially inserted in the skin layer 1 of a patient. During implantation, it is envisaged that this embodiment may extend partially from the skin layer. Where the electrically conductive core 2 extends past the normal skin layer of the patient, a layer of skin may be wrapped around the electrically conductive core 2 to effectively seal and form a barrier 6 between the electrically conductive core 2 and the external environment 18. In order to form the barrier 6 the patient's skin is wrapped around core 2 and stitched, thereby separating it from the external environment 18. After some time the wound at the point of insertion will heal and seal the core 2 from the external environment 18. This embodiment allows the electrically conductive core 2 to be fully implanted within the skin layer 1 in a manner that the electrically conductive core 2 is not required to perforate the skin layer 1, thereby minimising the risk of infection.

Preferably, a first coil 3 is threaded from the outside of the patient around the electrically conductive core 2 and the barrier 6 through the air gap 5 and may be connected back onto itself (not shown). This first coil 3 may also be wrapped partially or multiple times around the electrically conductive core 2 and the barrier 6 in a loose manner. The first coil 3 may then be connected to a controller or external power device (not shown). It will be appreciated by a person skilled in the art that the more windings forming the first coil 3, the higher the transmission efficiency of this embodiment.

Preferably, wrapped around or adjacent to the electrically conductive core 2 within the skin layer 1 is a second coil 4 which is in turn connected to internal wiring 7. The internal wiring 7 may be then connected to an implanted controller, battery or implantable active medical device (not shown).

The second coil 4 may also be encapsulated in a biocompatible material such as a biocompatible polymer, to limit the chance of reactions with the patient's body. Preferably, this encapsulation may be moisture resistant and hermetically sealed to prevent corrosion of the electrically conductive core 2.

Preferably, the surfaces around the air gap 5 may be reinforced with a protective material to prevent breakage of the device or injury to the patient. This is especially relevant in cases where the device is expected to be implanted for long term use. The protective material (not shown) may be placed over the barrier 6. This protective material protecting the conductive core may include: a biocompatible polymer, metal (for example titanium), Kevlar™ or similar material. This protective material may also form a protective moisture barrier inside the skin layer 1 and encapsulating the electrically conductive core 2. The protective material may also encapsulate barrier 6 to prevent chaffing.

Preferably, the electrically conductive core may be: constructed of a ferro-magnetic core encapsulated in a biocompatible material; and be rigid to support the formation of the air gap 5.

Preferably, polyurethane coated copper litz wire may be used for any windings around of either the first coil 3 or second coil 4. However, any suitable electrically conductive wire may be used. The multi-strand configuration of litz wire minimises the power losses otherwise encountered in comparable solid conductors or the tendency of radio frequency current to be concentrated at the surface of the conductor. Litz wire increases the surface area of the length of wire without significantly increasing the general size of the conductor.

Various other embodiments of TPDTS's in accordance with the present invention will described. In these embodiments the reference numerals for the skin layer 1, first and second coils 3, 4, barrier 6 and internal wiring 7, as used for the first embodiment will be maintained throughout the description.

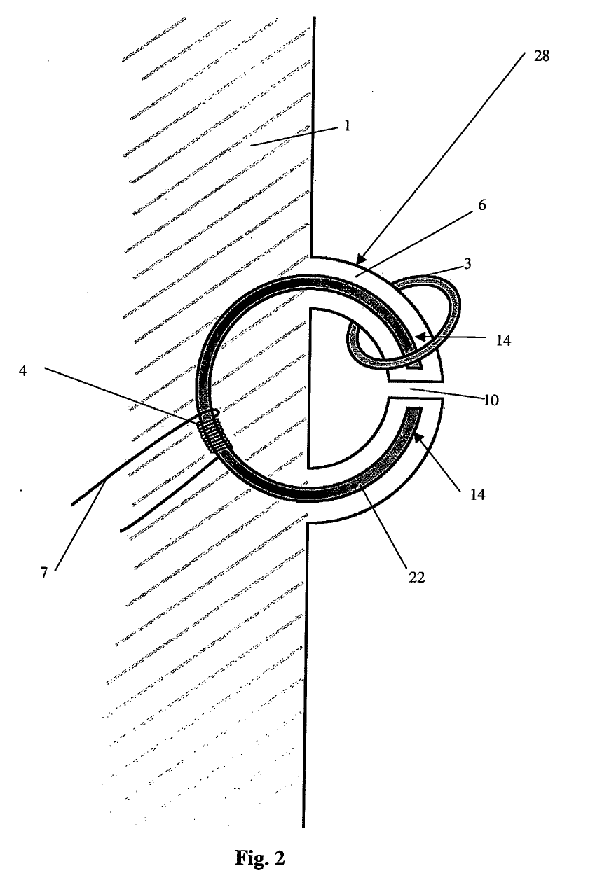

A second embodiment is shown in FIG. 2, wherein a TPDTS 28 comprises an electrically conductive core 22 that is generally C-shaped and a small additional air gap herein referred to as core gap 10.

The electrically conductive core 22 of this embodiment is preferably flexible which allows for its movement. Where the electrically conductive core 22 extends beyond the point at which the patient's normal skin layer would have been without this device, a barrier 6 similar to that of the first embodiment, encases the otherwise exposed electrically conductive core 22 and the protuberances 14. This barrier 6 is preferably constructed of skin material taken from the patient, but may also be constructed of other alternative materials such as biocompatible polymers. Barrier 6 is preferably flexible and resilient, and may also be constructed of entirely artificial means (not shown).

As a result of both the barrier 6 and the electrically conductive core 2 being preferably flexible, the protuberances 14 are also preferably flexible. This flexure may allow the patient, doctor or other user to bend or distort the shape of the protuberances 14 to facilitate the joining of a coupling device or the first coil 3, similar to that of the first embodiment, around the outer surface of the patient's skin which may allow transmission of an electric signal.

This embodiment has additional several benefits including: increased biocompatibility; improved ease of use by the patient; and reduced chance of injury the patient.

In FIG. 2, the core gap 10 may limit the effectiveness of the TPDTS 28 as it may increase the level of magnetic flux leakage. However, this embodiment may achieve a generally lower level of magnetic flux leakage than the prior art transcutaneous energy transmission systems.

Preferably, this embodiment may also include a latching means (not shown) to temporarily join the two outer free ends of the protuberances 14 to minimise the distance of core gap 10. This latching means may be preferably achieved by the insertion of small positioning magnets at opposed ends of the protuberances 14. However, alternate forms of latching means are available and these may include: mechanical clips or sticky re-useable glue.

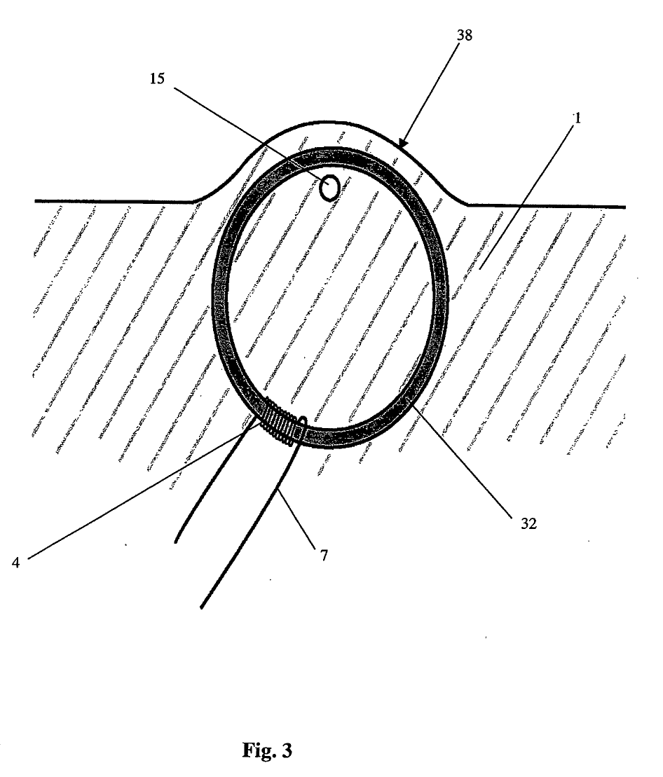

A third embodiment similar to that of FIG. 1 is shown in FIG. 3 however the TPDTS 38 has an elliptical ring shaped core 32. A hole 15 is made in skin layer 1 so that it passes under the outer most edge of the electrically conductive core 32. This hole 15 replaces the function of the air gap 5 of the first embodiment, and allows for the attachment of a first coil (not shown in FIG. 3). The first coil, in use, passes through the hole 15 and allows for communication of electrical signals to and from the second coil 4.

The implantation of this third embodiment may be a staged procedure in which the electrically conductive core 32 is inserted. After the insertion wound has healed, the hole 15 may be formed by a punch mechanism or the insertion of a needle or a sharpened piece of wire. The hole 15 may be relatively small relative to the air gap 5 of the first embodiment, and this may further reduce the likelihood of infection. In a manner similar to piercing ear lobes, a degree of healing may occur around hole 15 thus reducing the likelihood of infection.

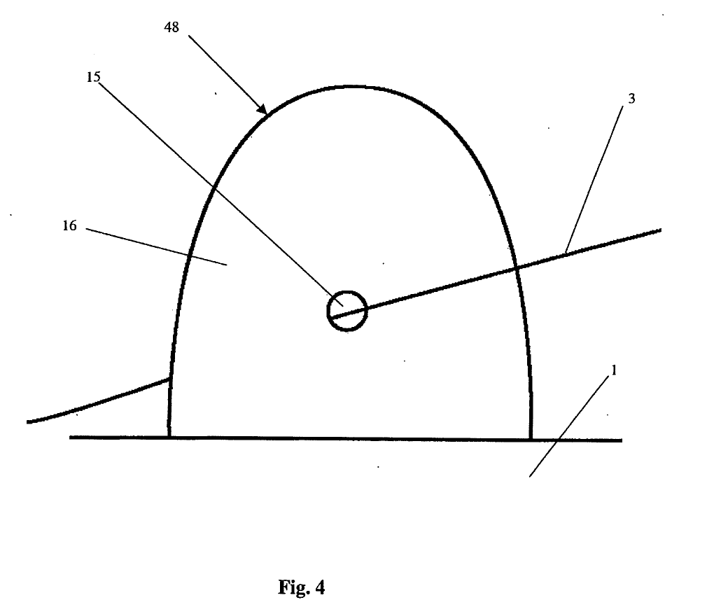

A fourth embodiment is shown by FIGS. 4 & 5. FIG. 4 shows a TPDTS 48 working in conjunction with a flat flap of skin 16 protruding from the surface of the patient's ordinary skin layer 1.

The implantation of this embodiment may be achieved by pinching and possibly sewing a flap of skin 16 and inserting the electrically conductive core 42 within the flap of skin 16. Alternatively, implantation may also take advantage of existing anatomical flap (such as an earlobe of a patient) which may be utilised. The flap of skin 16 may also have the benefit of making the hole 15 comparatively short so that re-epithelialisation of the tunnel would be facilitated reducing the likelihood of infection and the hole 15 may be created with the use of a surgical needle.

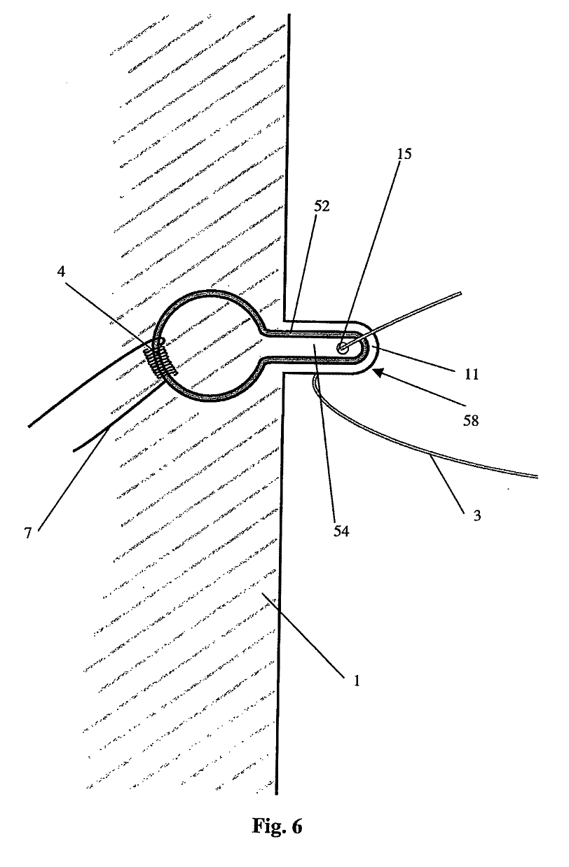

In a fifth embodiment as depicted in FIG. 6, a TPDTS 58 includes a bent or deformed electrically conductive core 52. This may provide an improved anchoring means within the skin layer 1. The upper end of the electrically conductive core 52 has a single protuberance 54.

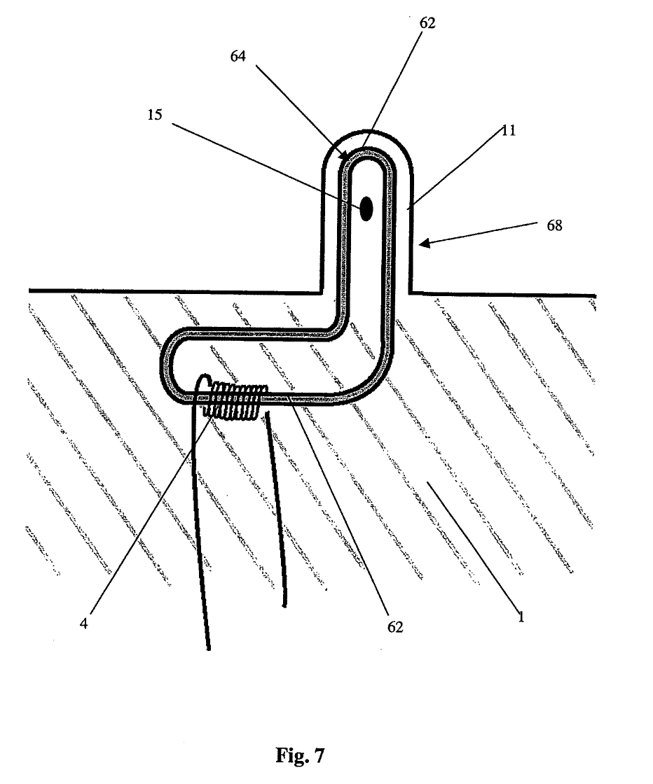

In a sixth embodiment as depicted in FIG. 7 a TPDTS 68 includes a bent or deformed electrically conductive core 62. The upper end of electrically conductive core 62 has a single protuberance 64.

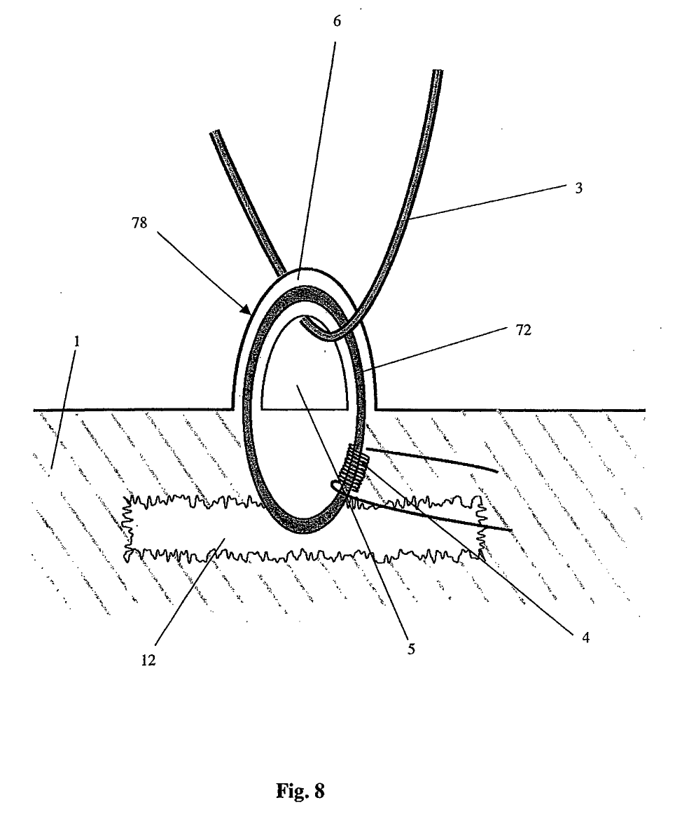

In FIG. 8, a seventh embodiment of a TPDTS 78 is shown. A subcutaneous textured surface 12 has been used to anchor or maintain the electrically conductive core 72 in the correct shape and orientation. The textured surface 12 may also cover the entire or a portion of the surface of electrically conductive core 72 facilitating tissue ingrowth and mechanical stabilisation (not shown in FIG. 8).

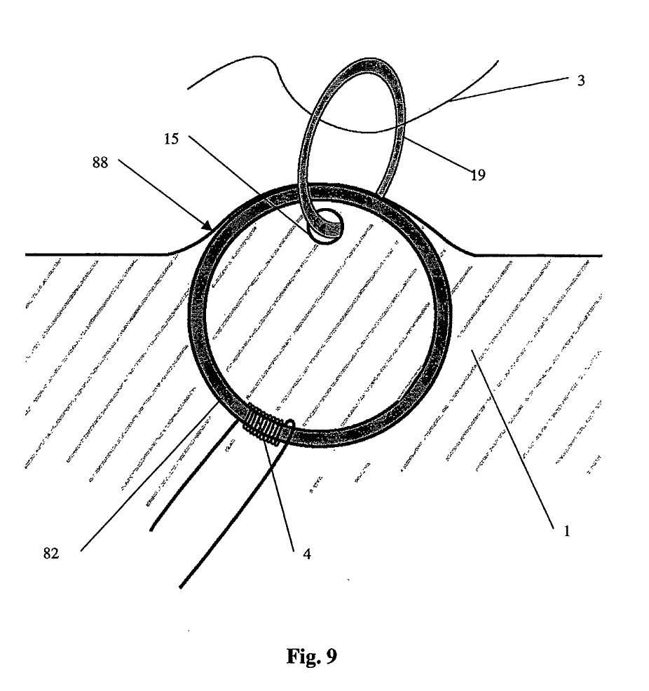

In FIG. 9, an eighth embodiment of a TPDTS 88 is shown, having an electrically conductive core 82. A “sleeper” ring 19 is included, similar to an earring, to help maintain the hole 15 and reduce abrasive wear from the first coil 3. The sleeper ring 19 may preferably be made of gold, silver or similar material which is known to work well in maintaining the hole 15 with relatively low infection rates.

In circumstances where damage or infection to the skin are likely, a backup ring (not shown) may be implanted distant from the primary ring or alternately, a temporary primary wire may be subcutaneously tunnelled through a deeper part of the electrically conductive core using a surgical needle.

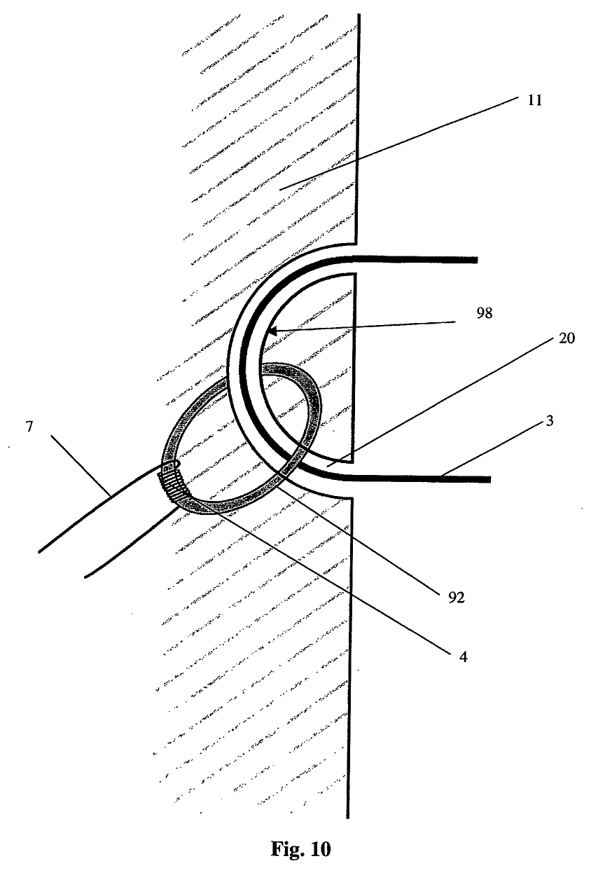

In FIG. 10, a ninth embodiment of a TPDTS 98 is shown having an electrically conductive core 92. A U-shaped tunnel 20 is created in the skin layer of a patient 1 through the centre of electrically conductive core 92. A wire may be passed through the generally U-shaped tunnel 20 to form the first coil 3. Preferably, the wire may be textured (to promote tissue incorporation) and thin. This may provide a stable biological interface which may be less subject to infection than a standard percutaneous lead.

Whilst in operation of the abovementioned embodiments, an electrical signal may also be delivered through first coil 3. The electrical signal in turn generates an electromagnetic force (‘EMF’) radially from the surface of the first coil 3. At the point where the electrically conductive core cuts said EMF, another EMF is generated within the electrically conductive core. The electrically conductive core then passes this EMF to the second coil 4 and an electrical signal is then induced in said second coil 4. Second coil 4 may then pass the electrical signal to an internal controller, a rectifier, an internal battery or other implantable active medical devices (not shown) by internal wiring 7.

The electrical signal may be bidirectional and may be either transmitted and received by first 3 or second coil 4. This electrical signal may be at full duplex or half duplex depending on the circuit requirements.

Preferably a direct current (DC) rectifier (not shown) or multiple rectifiers (not shown) may be added to the abovementioned embodiments. These rectifiers may be added to filter or condition the electrical signal received and/or transmitted by both first coil 3 and second coil 4.

The aforementioned embodiments may be used with various active implantable medical devices. These may include: rotary blood pumps, heart pumps, and left ventricle assist device.

The electrical signal transmitted by these embodiments may include: power; and/or data information. Said data information may include: software controller upgrades, data regarding patient health, status of the medical device; and control signals.

The above descriptions only describes some of the embodiment of the present inventions and modification, it may be obvious to those skilled in the art that further modifications can be made thereto without departing from the scope and spirit of the present invention.

1. A device for communicating electric signals across the skin layer of a patient, wherein said device includes: an electrically conductive core capable of forming an EMF flux loop; first and second coils, which are in EMF communication with said electrical conductive core and wherein said first coil is positioned externally to said patient and surrounds at least a first portion of said electrically conductive core; and said second coil is implanted beneath or in said skin layer and surrounds at least a second portion of said electrically conductive core.

2. The device as claimed in claim 1, wherein said electrically conductively core is implanted at least partially within said skin layer.

3. The device as claimed in claim 1, wherein said electrically conductively core is formed in a loop or ring-like configuration.

4. The device as claimed in claim 3, wherein said electrically conductively core does not breach an outer surface of said skin layer.

5. The device as claimed in claim 1, wherein said device includes a sleeper ring to interact with said first coil.

6. The device as claimed in claim 1, wherein said device includes a textured surface on at least a portion of said electrically conductively core.

7. The device claimed in claim 1, wherein said electrically conductive core is encapsulated within said skin layer.

8. The device as claimed in claim 1, wherein said device includes a layer of protective material surrounding at least a portion of the electrically conductive core.
